(19) United States
(12) Patent Application Publication
(10) Pub. No.: US 2006/0222144 A1
(43) Pub. Date: Oct. 5, 2006

(54) Method for operating a computed tomography apparatus to adjust attenuation valves from respective radiation detector elements

(76) Inventor: Johann Russinger, Buckenhof (DE)

Correspondence Address: SCHIFF HARDIN LLP;Patent Department, 6600 Sears Tower, 233 South Wacker Drive, Chicago, IL, 60606, US

(21) Appl. No.: 11/347,168

(22) Filed: Feb. 3, 2006

(30) Foreign Application Priority Data
Feb. 4, 2005 (DE) 10 2005 005 285.1

(51) Int. Cl. H05G 1/60; A61B 6/00; G01N 23/00; G21K 1/12

(52) U.S. Cl. 378/16

(57) ABSTRACT

In a method for operating a computed tomography apparatus for scanning a subject, a characteristic value is determined only from the attenuation values of selected detector elements of a detector on which x-rays are incident that penetrated the examination region of the subject. An adjustment value for adjustment of the intensity of the x-ray radiation is calculated for the following projection from at least one such determined characteristic value. The intensity of the x-ray radiation is adjusted for a following projection dependent to the calculated adjustment value. The method prevents image artifacts (in particular over-exposed or under-exposed image regions) in a resulting image calculated from the attenuation values, such as in a calculated slice or volume image of an examination region.

### BACKGROUND OF THE INVENTION

1. Field of the Invention

The present invention concerns a method for operating a computed tomography apparatus having an acquisition system for scanning a subject, whereby the subject having an examination region and the acquisition system having a measurement region, these regions being displaceable relative to each other, and the acquisition system including an x-ray radiator that emits x-ray with an adjustable intensity and a detector formed by a number of detector elements for acquisition of attenuation values of a projection.

2. Description of the Prior Art

A method for operating a computed tomography apparatus having an acquisition system with an x-ray radiator and a detector is known from DE 198 07 639 C2, in which the intensity of the x-ray radiation emanating from the x-ray radiator is continuously adjusted such that a patient is exposed to an optimally low x-ray dose during an examination. A maximum attenuation per projection is determined from all attenuation values acquired by a detector. A maximum attenuation value is extrapolated for the following projection on the basis of a number of determined maximum attenuation values of previous projections. The adjustment of the intensity of the x-ray radiation for the following (next) projection subsequently ensues dependent on the extrapolated maximum attenuation value.

This calculation of the extrapolated attenuation value, and thus the adjustment of the intensity of the x-ray radiation, ensues independently of the current position of the measurement region of the acquisition system and the current position of the examination region (of the patient) relative to one another. In operating modes of the computed tomography apparatus in which the examination region and the measurement region are shifted relative to one another in the direction of a system axis of the computed tomography apparatus, there is a risk that the determined attenuation values or the adjusted intensities of the x-ray radiation are not always optimally matched to the examination region of the patient. This can lead to a reduction of the achievable image quality of an image determined from the attenuation values of the examination region, such that a diagnosis by a treating doctor on the basis of the generated image information is made more difficult.

### SUMMARY OF THE INVENTION

An object of the present invention is to provide a method for operating a computed tomography apparatus of the type described above, wherein the achievable image quality of a resulting image (diagnostic image) that can be calculated from attenuation values is improved.

The invention is based on the recognition that the adjustment of the intensity of the x-ray radiation in the known apparatus can occur in a disadvantageous manner due to the use of attenuation values that were acquired outside of the examination region of the subject. Attenuation values outside of the examination region can differ significantly from the attenuation values within the examination region.

Different attenuation values within and outside of the examination region are particularly to be expected when essentially only soft tissue is present within the examination region, and essentially only osseous (bony) tissue is present in the adjoining subject region.

In the border regions of the image calculated from the attenuation values of the examination region, this situation can lead to a reduction of the achievable image quality. For example, in the boundary regions of the image, images oriented perpendicularly to the feed direction of a subject to be examined can be either under-exposed or over-exposed. Such image errors are prevented with the present invention.

The above object is achieved by a method for operating a computed tomography apparatus for scanning a subject in accordance with the invention wherein a characteristic value is determined from the attenuation values of selected detector elements that acquire the x-ray radiation that penetrated through the examination region, and an adjustment value for adjustment of the intensity of the x-ray radiation for the following projection is calculated from at least one determined characteristic value of an earlier projection, and the intensity of the x-ray radiation is adjusted for a following projection dependent on the calculated adjustment value.

The adjustment of the intensity of the x-ray radiation thus always ensues on the basis of attenuation values that originate from the examination region of the subject to be examined. Attenuation values outside of the examination region are not used. The adjustment of the intensity of the x-ray radiation thus always ensues such that the attenuation values acquired by the detector exhibit a dynamic range, so that under-exposure or over-exposure of image regions of a calculated result image is avoided.

Upon entrance of the examination region of the subject into the measurement region of the acquisition system, the characteristic value is taken from the attenuation values of selected detector lines (formed by the detector elements) that already have acquired x-ray radiation that penetrated the examination region of the subject.

The adjustment of the intensity of the x-ray radiation is thus independent of the subject regions adjoining the examination region, such that the resulting images (for example slice or volume images) determined from the attenuation values do not exhibit an over-exposed or under-exposed image region.

Over-exposed or under-exposed image regions of the resulting image also are prevented by not only upon entrance, but also upon exit of the examination region from the measurement region, the characteristic value is determined from the attenuation values of selected detector lines that are still acquiring radiation from the examination region of the subject.

The adjustment of the intensity of the x-ray radiation thus is also effected upon exit, based only on attenuation values which originate from the examination region. An unintentional under-exposure or over-exposure of image regions of the result image is avoided.

In an embodiment of the invention, the adjustment value for adjustment of the intensity of the x-ray radiation is calculated from at least one characteristic value of an earlier projection of the same rotation of the acquisition system. The calculation of an adjustment value on the basis of characteristic values of the same projection enables a very fast adaptation of the intensity of the x-ray radiation (for example already after a small number of projections) to the attenuation properties of the examination region of the subject. This is particularly advantageous in the first rotation of the acquisition system around the subject, in which characteristic values have not yet been determined at all angle positions of the rotation.

The adjustment value also can be calculated from at least one characteristic value of an earlier projection of the same projection angle from different rotations of the acquisition system. Projections that are acquired from the same projection angle radiate through the subject in the same manner given a slight feed of the examination region in the direction of the rotation axis of the computed tomography apparatus. The characteristic values of earlier projections that are thus determined enable a very precise adjustment of the intensity of the x-ray radiation dependent on the projection angle or dependent on the geometry with which a subject is irradiated by the x-ray radiation.

The calculation of the adjustment value can also include an extrapolation of the determined characteristic values, such that the adjustment of the intensity of the x-ray radiation ensues on the basis of an estimated value of the characteristic value of the following projection.

The extrapolation preferably is a linear extrapolation technique of the first order, such that the calculation of the adjustment value requires a small calculation time and thus can be effected in real time (online) with each projection.

As an alternative, the extrapolation can be a linear technique of the second order that, given with slight additional outlay, enables the calculation of the adjustment value with a smaller estimation error of the aforementioned characteristic value.

Other extrapolation methods can be used that, for example, are based on polynomial approximations, Taylor series, spline interpolations or methods of the nth order.

The characteristic value preferably is a maximum attenuation value of the acquired attenuation values of selected detector elements. The determination of the maximum attenuation value requires a small calculation time and therefore can be calculated in real time with each projection.

Conventional x-ray tubes can be used in the x-ray radiator in accordance with the invention, so the method can be executed without substantial re-design of the structure of the computed tomography apparatus.

The intensity of the x-ray radiation can be adjusted in a simple manner using an adjustment value in the form of an x-ray tube voltage. As an alternative, the intensity of the x-ray radiation can be adjusted by an adjustment value in the form of an x-ray tube current.

### DESCRIPTION OF THE DRAWINGS

FIG. 1 is a schematic block diagram of a computed tomography apparatus for execution of the inventive method.

FIG. 2 shows an examination region of a patient upon entrance into a measurement region of the acquisition system of the apparatus of FIG. 1.

FIG. 3 shows the examination region of FIG. 2 that has become completely moved into the measurement region of the acquisition system.

FIG. 4 shows the examination region of FIG. 3, wherein a part of the examination region is outside of the measurement region upon exit from the measurement region.

FIG. 5 illustrates extrapolation of a characteristic value with an extrapolation technique of the first order.

FIG. 6 illustrates extrapolation of a characteristic value with an extrapolation technique of the second order.

### DESCRIPTION OF THE PREFERRED EMBODIMENTS

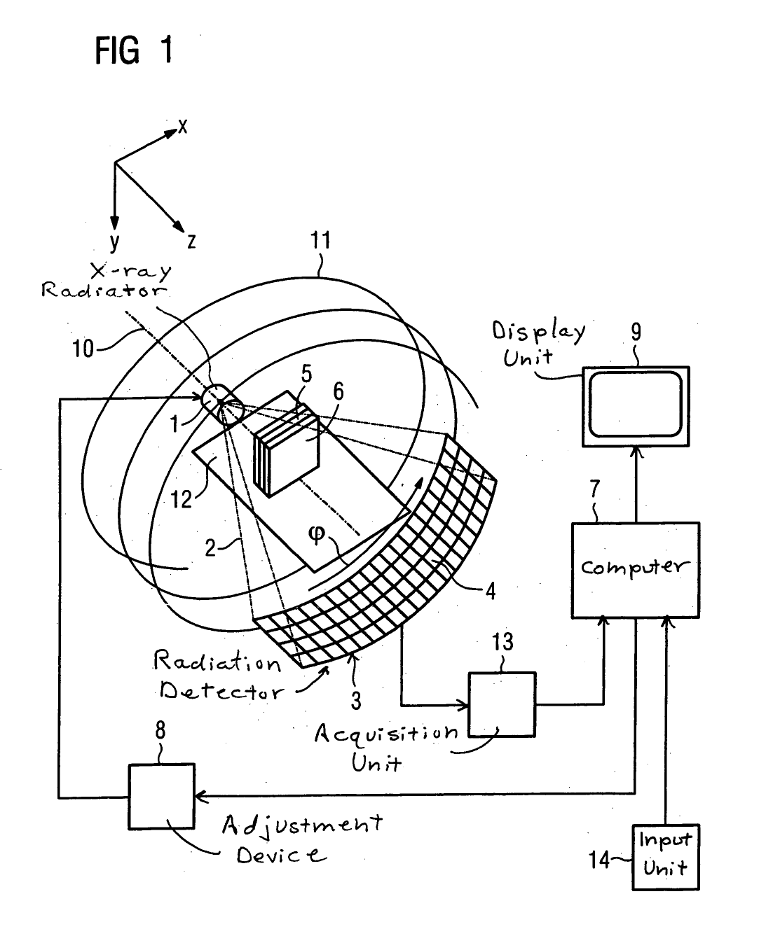

The computed tomography apparatus as shown in FIG. 1 has a movable table plate 12 by means of which an examination region 5 of a subject 6 (for example the examination region of a patient) to be examined can be moved through an opening in the housing of the computed tomography apparatus into a measurement region (data acquisition region) 2 of an acquisition system of the computed tomography apparatus. The examination region 5 of the subject 6 and the measurement region 2 of the acquisition system can be displaced relative to one another.

The acquisition system includes,an x-ray radiator 1 (for example an x-ray tube) and a detector 3 arranged opposite the radiator 1, the detector 3 being composed of a number of detector elements 4 arranged in columns and rows. The x-ray radiator 1 generates a fan-shaped x-ray beam. This x-ray beam penetrates the measurement region 2 of the acquisition system and strikes the detector elements 4 of the detector 3. Each detector element 5 generates an attenuation value dependent on the attenuation of the x-ray radiation passing through the measurement region 2 and incident thereon. The conversion of the x-ray radiation into attenuation values ensues, for example, by means of a photodiode optically coupled with a scintillator, or by means of a directly-converting semiconductor. A set of attenuation values of the detector 3 that are acquired for a specific position of the x-ray radiator 1 relative to the subject 6 is known as a “projection”.

A gantry (not shown) on which the acquisition system is mounted is located inside the computed tomography apparatus. The gantry is rotated by a drive unit (not shown) with a high rotation speed around the system axis 10 of the computed tomography apparatus in a known manner. A number of projections of the subject 6 can be produced from different angle positions in this manner. In particular, an examination region 5 of the subject 6 that is larger than the measurement region 2 of the acquisition system can be scanned by a rotation of the gantry given simultaneous, continuous feed of the subject 6 in the direction of the system axis 10. The projections acquired from such a spiral scan 11 used to calculate slice or volume images that are visually presented on the display unit 9 to the operating personnel.

The inventive method is applicable for not only spiral scanning 11 of a subject 6, but also is applicable to scan a subject 6 without rotation, merely with only a feed of the subject 6 in the direction of the system axis 10. For example, this operating mode enables the generation of an overview image in the form of a to program. Moreover, the method can be used without feed, with only rotation of the acquisition system. It is only of importance that only those attenuation values that are associated with the examination region 5 of the subject 6 in every projection be used in the determination of the characteristic value. In this case, the adjustment of the intensity of the x-ray radiation leads to no artifacts whatsoever, i.e. no under-exposure or over-exposure whatsoever of image regions of the resulting image.

The computed tomography apparatus additionally has the following system components: an acquisition unit 13 for acquisition of the attenuation values generated by the detector 3, a computer 7 for determination of the characteristic value on the basis of attenuation values of selected detector elements 4 and for calculation of an adjustment value of the intensity of the x-ray radiation, an adjustment device 8 for adjustment of the intensity of the x-ray radiation on the basis of the calculated adjustment value, and an input unit 14 for input of operating and examination parameters.

The quality of the resulting image calculated from the projections, for example a slice or volume image, is primarily dependent on quantum noise of the detector elements 4 that is associated with the x-ray dose that is used for acquisition of the attenuation values. Image artifacts due to noise then become increasingly noticeable, either because the applied x-ray dose is too low or the intensity of the x-ray radiation was too strongly attenuated, for example due to the anatomy of the patient. In order to keep the attenuation values generated by the detector elements 4 above the noise level, the intensity of the x-ray radiation therefore must be continuously adapted during the examination of the examination region 5 of the subject 6. In known methods for this purpose the maximum attenuation value is determined per projection from all attenuation values acquired by the detector 3. The attenuation value of a following projection is subsequently predicted from at least one stored attenuation value, such that an adaptation of the intensity of the x-ray radiation can ensue for the following projection.

It is unavoidable that upon entrance and upon exit of the examination region 5 of the subject 6 into and out from the measurement region 2 of the acquisition system attenuation values are also acquired that lie outside of the examination region 5. Dependent on the attenuation properties of the subject regions situated around the examination region 5, it is possible that the determined maximum attenuation value therefore originates from a detector element on which radiation was incident that did not penetrate the examination region 5, but instead passed through an adjoining region. Adjustment of the intensity of the x-ray radiation on the basis of such an attenuation value leads to the resulting image exhibiting image regions that are either under-exposed or over-exposed. This reduction of the image quality is noticeable, for example, in the scanning of an organ surrounded by bones, for example in the scanning of the kidneys, which abut on a pelvic bone of the patient.

The inventive method prevents such losses in the image quality by insuring that only attenuation values from those detector elements 4 on which x-rays are incident that penetrated the examination region 5 are used for the determination of a characteristic value (for example of a maximum attenuation value).

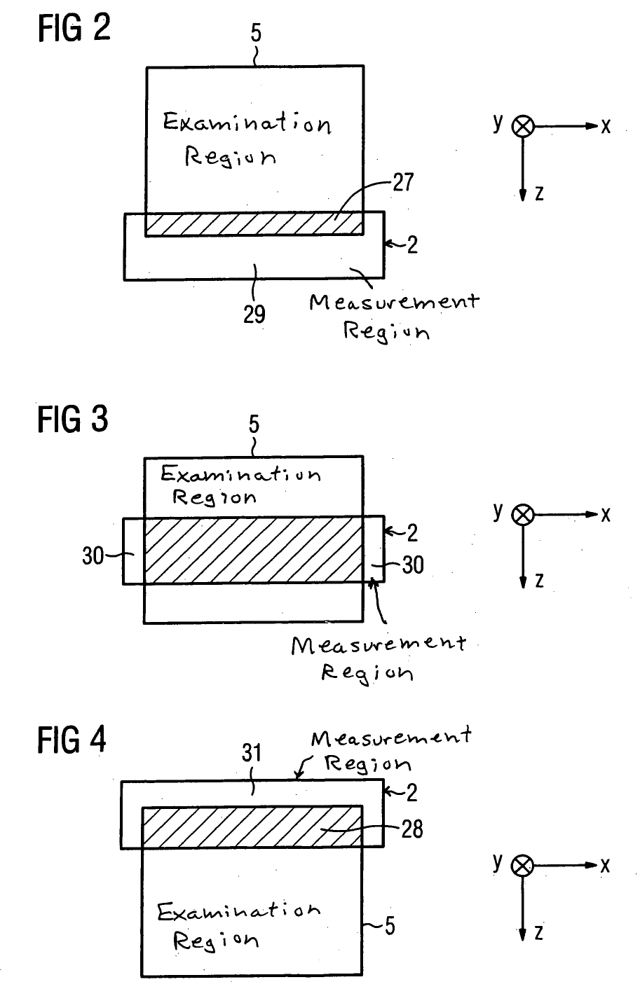

The following FIGS. 2 through 4 are examples of various positions of the examination region 5 of the patient relative to the measurement region 2 of the acquisition system 1, 3, in which the inventive method for prevention of under-exposed or over-exposed image regions in the resulting image is advantageous.

The position of the examination region 5 upon entrance into the measurement region 2 of the acquisition system is shown in FIG. 2. The measurement region 2 of the detector 3 acquires not only a part 27 of the examination region 5, but also a part 29 of a subject region adjoining the examination region. The determination of the characteristic value, however, ensues only on the basis of those attenuation values that originate from detector elements that cover the examination region 5.

The examination region 5 is completely located in the measurement region 2 of the detector 3 in FIG. 3. In the shown example, the coverage of the measurement region 2 is, however, greater than the region that is accepted by the examination region 5 given scanning with the acquisition system such that attenuation values are acquired in the boundary region of the measurement region 2 from subject regions 30 that do not belong to the examination region 5. The attenuation values generated by these detector elements 4 are not used, since only these attenuation values that are situated within the examination region 5 should make a contribution to the determination of the characteristic value.

FIG. 4 shows the exit of the examination region 5 from the measurement region 2 of the detector 3. The detector 3 acquires (in a manner identical to that of FIG. 2) not only a part 28 of the examination region 5, but also a part 31 of the subject region adjoining the examination region 5. In this case, the determination of the characteristic value also ensues on the basis of those attenuation values that originate from detector elements 4 which cover the examination region 5.

For example, in the shown cases the selection of the detector elements 4 that acquire the examination region 5 ensues dependent on the rotation angle and on the basis of the examination parameters established before the beginning of the examination. The examination parameters are set by an operator via the input unit 14 and include, for example, geometric data about the volume of the examination region to be scanned, and/or the relative position relation between the examination region and the measurement region, and/or a set pitch that establishes the amount of feed of the examination region in the direction of the system axis 10 of the computed tomography apparatus per rotation of the acquisition system.

The determination of the characteristic value alternately can ensue in real time for each projection or only for projections at angle positions that were previously determined. The predetermined angle positions, for example, can exhibit an angle interval relative to one another that has been predetermined. This can be advantageous for calculation time reasons, such that the determination of the characteristic value can be implemented in real time from all relevant attenuation data for the entire examination.

The determined characteristic values are stored and subsequently serve for calculation of an extrapolated characteristic value from which an adjustment value is calculated for adjustment of the intensity of the x-ray radiation. In a simple variant of the invention described in detail here, the characteristic value is the maximum attenuation value of a projection. In the following, the term “maximum attenuation value” is therefore used instead of the term “characteristic value”.

The calculation of the adjustment value for adjustment of the intensity of the x-ray radiation ensues in an anticipatory manner for the following projection on the basis of at least one determined and stored maximum attenuation value, and involves an extrapolation of the maximum attenuation value for the following projection.

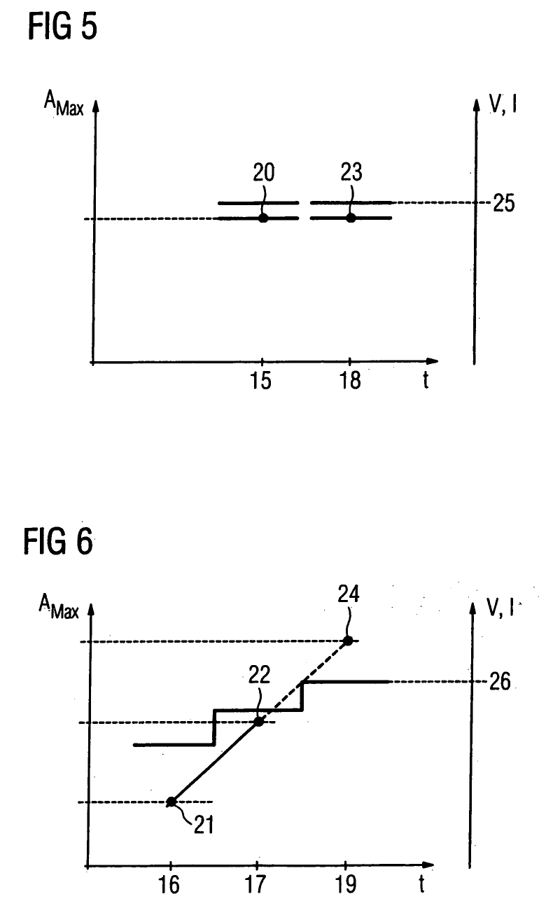

The simplest extrapolation (and therefore the extrapolation that can be realized in a very efficient manner) of the maximum attenuation value is a method of the first order (shown in FIG. 5), which assumes that the maximum attenuation value 23 of the intensity of the x-ray radiation of the following projection 18 is approximately equal to the maximum attenuation value 20 of the intensity of the x-ray radiation of the earlier projection:
ApredMax=AMax(t−1)
wherein ApredMax is the extrapolated maximum attenuation value 23 and AMax (t−1) is the determined maximum attenuation value 20 of the last acquired earlier projection 15. Depending on the design of the method, the last acquired earlier projection 15 is, for example, that projection that was acquired within the same rotation at a different angle position previously adopted by the acquisition system. In this case, the extrapolation of the maximum attenuation value is implemented in the direction of the rotation movement of the acquisition system (thus along the (φ-axis) from previously-acquired maximum attenuation values of adjacent rotation angle positions. Alternatively, the last acquired projection can be a projection that was acquired at the same projection angle, but originates from a different, earlier rotation of the acquisition system. In this case, the extrapolation of the maximum attenuation value is implemented in the direction of the rotation axis of the computed tomography apparatus (thus along the z-direction) from previously acquired maximum attenuation values, with the maximum attenuation values that are used for extrapolation originating from earlier rotations of the acquisition system, but acquired at a nearly identical rotation angle position of the acquisition system. These two embodiments of the method can be advantageously used in combination.

For example, it is possible for the extrapolation to ensue during the first rotation on the basis of the determined maximum attenuation values that originate from the same rotation and that were acquired at adjacent rotation angle positions. For the first rotation, namely, a maximum attenuation value is not available for the same angle position of the acquisition system from an earlier rotation. As an alternative to this approach, it is possible for the extrapolation of the maximum attenuation value to be implemented for the first rotation on the basis of maximum attenuation values that originate from regions that are immediately adjacent to the examination region. In this case, the extrapolation would already ensue in the direction of the system axis for the first rotation. This provides good expected values for the maximum attenuation values when the subject exhibits nearly identical attenuation properties in the immediate surroundings of the examination region. In any case, subsequent to the first rotation it is possible, in all following projections, for the maximum attenuation value to be extrapolated on the basis of the determined maximum attenuation values from different rotations at the same position, such that an adaptive adjustment of the intensity of the x-ray radiation is ensured for all projections.

The adjustment value 25 for adjustment of the x-ray radiation is established on the basis of the extrapolated maximum attenuation value 23, such that the determined maximum attenuation value for the following projection is optimally close to the extrapolated attenuation value.

As shown in FIG. 6 the maximum attenuation value alternatively can be advantageously extrapolated in an anticipatory manner with a method of the second order. In this method, the determined maximum attenuation values of the two last projections are used:
ApredMax=2*AMax(t−1)−AMax(t−2)
wherein ApredMax is the extrapolated maximum attenuation value 24, and whereby AMax(t−1) and AMax(t−2) are the maximum attenuation values 21, 22 of both of the last acquired earlier projections 16, 17. In this case it is also possible for the last acquired earlier projections 16, 17 to originate from the same rotation at different angle positions of the acquisition system or from successive rotations given approximately identical angle positions of the acquisition system.

The adjustment value 26 for adjustment of the intensity of the x-ray radiation is calculated on the basis of the extrapolated maximum attenuation value 24, such that in the following projection the maximum attenuation value determined there optimally precisely corresponds to the extrapolated attenuation value 24.

The adjustment value 26 can be the x-ray current of the x-ray tube. The relation between the extrapolated maximum attenuation value 24 and the adjustment value 26 can be stored in the form of a look-up table, for example, such that the calculation of the adjustment value 26 is reduced to the readout of a value entered into the table.

The calculated adjustment value 26 is transmitted to the control unit 8 that is connected with the x-ray radiation detector 1, and effects the adjustment of the x-ray radiator 1 for the following projection. The adjustment of the x-ray current (and thus the adjustment of the intensity of the x-ray radiation) can be very slow, such that additional corrective measures may be necessary with which the desired intensity can be adjusted. For example, to achieve a desired intensity the x-ray current can be temporarily varied in the form in the form of a predeterminable current profile using the value predetermined by the adjustment value, such that the desired intensity of the x-ray radiation is achieved in the following projection.

As an alternative, it is possible for the adjustment value to be an x-ray tube voltage. X-ray tubes with an additional grid cathode enable a very fast modulation of the intensity of the x-ray radiation on the basis of an adjustable x-ray tube voltage.

The method described herein is, not limited to the use of an x-ray tube. It is applicable as well to an x-ray radiator with a different type of x-ray source, in which case other adjustment values are used for adjustment of the intensity.

Although modifications and changes may be suggested by those skilled in the art, it is the intention of the inventor to embody within the patent warranted hereon all changes and modifications as reasonably and properly come within the scope of his contribution to the art.

I claim as my invention:

1. A method for operating a computed tomography apparatus having an image data acquisition system comprising a radiation source that emits radiation having a radiation intensity, and a radiation detector on which radiation is incident, said radiation detector comprising a plurality of detector elements with each detector element generating an attenuation value representing said radiation incident thereon, said image data acquisition system having a measurement region and being adapted to interact with a subject, having an examination region, to acquire image data from the examination region during a relative displacement between said measurement region and said examination region, said method comprising the steps of:
automatically electronically identifying selected detector elements, from among said plurality of detector elements, on which said radiation is incident that penetrated through the examination region in a current irradiation of the examination region;
from at least one of said characteristic values, automatically electronically determining an adjustment value for adjustment of said radiation intensity; and
in a subsequent irradiation of the examination region following said current irradiation, automatically electronically adjusting the intensity of the radiation emitted in said subsequent irradiation dependent on said adjustment value.

2. A method as claimed in claim 1 wherein said detector elements are arranged in respective detector lines of said radiation detector and wherein said method comprises, upon entrance of said examination region into the measurement region, selecting said selected detector elements by selecting selected detector lines on which said radiation is incident that has penetrated through the examination region.

3. A method as claimed in claim 1 wherein said detector elements are arranged in respective detector lines of said radiation detector and wherein said method comprises, upon exit of said examination region into the measurement region, selecting said selected detector elements by selecting selected detector lines on which said radiation is still incident that has penetrated through the examination region.

4. A method as claimed in claim 1 wherein said radiation source is rotated around said subject to irradiate said subject in successive projections, and wherein said current irradiation is a current projection of a rotation of said radiation source around the subject and wherein said: subsequent irradiation is a subsequent projection in the same rotation.

5. A method as claimed in claim 1 wherein said radiation source is rotated multiple times around said subject while irradiating said examination region from at least one projection angle in each rotation, and wherein said current irradiation is a projection at a projection angle in one of said multiple rotations and wherein said subsequent irradiation is a projection at the same projection angle in a different one of said multiple rotations.

6. A method as claimed in claim 1 comprising automatically electronically determining said adjustment value by computerized extrapolation of a plurality of said characteristic values.

7. A method as claimed in claim 6 comprising employing a linear extrapolation algorithm of the first order as said computerized extrapolation.

8. A method as claimed in claim 6 comprising employing a linear extrapolation algorithm of the second order as said computerized extrapolation.

9. A method as claimed in claim 1 comprising employing, as said at least one characteristic value, a maximum of the respective attenuation values of the selected detector elements.

10. A method as claimed in claim 1 comprising employing an x-ray radiator having an x-ray tube as said x-ray source to emit x-ray radiation as said radiation.

11. A method as claimed in claim 10 wherein said x-ray tube operates with a tube voltage, and comprising employing said adjustment value to adjust said tube voltage to adjust said radiation intensity.

12. A method as claimed in claim 10 wherein said x-ray tube operates with a tube current, and comprising employing said adjustment value to adjust said tube current to adjust said radiation intensity.
